(19) United States
(12) Patent Application Publication
(10) Pub. No.: US 2019/0331698 A1
(43) Pub. Date: Oct. 31, 2019

(54) MOLECULAR MARKER AND KIT FOR EARLY DIAGNOSIS AND PREDICTION OF SEPSIS ACUTE KIDNEY INJURY AND APPLICATION THEREOF

(71) Applicant: THE SECOND XIANGYA HOSPITAL OF CENTRAL SOUTH UNIVERSITY, Hunan (CN)

(72) Inventors: Dongshan ZHANG, Hunan (CN); Siyuan QU, Hunan (CN); Huiling LI, Hunan (CN); Pan ZHANG, Hunan (CN)

(73) Assignee: THE SECOND XIANGYA HOSPITAL OF CENTRAL SOUTH UNIVERSITY, Hunan (CN)

(21) Appl. No.: 16/405,992

(22) Filed: May 7, 2019

Related U.S. Application Data
(63) Continuation of application No. PCT/CN2018/086671, filed on May 14, 2018.

(30) Foreign Application Priority Data
May 19, 2017 (CN) 201710358057.2

(51) Int. Cl. G01N 33/68; C12Q 1/686

(52) U.S. Cl. CPC G01N 33/6893; C12Q 1/686; G01N 2800/52; G01N 2800/50; G01N 2800/347

(57) ABSTRACT

The present invention relates to a molecular marker, a kit and an application for early diagnosis and prediction of sepsis acute kidney injury (AKI). TCONS_00024536 is highly expressed in the blood of sepsis AKI patients. By detecting the expression level of TCONS_00024536 in the blood of patients, whether the patients suffer from sepsis AKI or the probability of suffering from sepsis AKI can be judged. The present invention is of great significance for specific and sensitive early diagnosis and prediction of sepsis AKI.

### CROSS-REFERENCE TO RELATED APPLICATION

This application is a continuation of international PCT application serial no. PCT/CN2018/086671, filed on May 14, 2018, which claims the priority benefit of Chinese application no. 201710358057.2, filed on May 19, 2017. The entirety of each of the above-mentioned patent applications is hereby incorporated by reference herein and made a part of this specification.

### BACKGROUND

### Technical Field

The present invention relates to the technical field of molecular diagnosis, in particular to a molecular marker and a kit for early diagnosis and prediction of sepsis acute kidney injury and application thereof.

### Description of Related Art

Sepsis is one of the common causes and complications of critically ill patients, and the mortality caused thereby is 50% or above. Meanwhile, sepsis is also a common cause of acute kidney injury in severe patients. Once sepsis patients have acute kidney injury, the mortality can reach 70%. Therefore, early diagnosis and treatment are the key of reducing the morbidity and mortality of sepsis-related AKI. At present, the diagnosis of acute kidney injury mainly depends on serum creatinine and urine volume. However, serum creatinine and urine volume are affected by various factors, so renal function changes cannot be accurately reflected in time and there is not sufficient sensitivity and specificity for AKI diagnosis. It is now known that creatinine concentration changes only when renal function loss reaches 50%, and it takes several days for creatinine to reach a steady state, so renal functions cannot be reflected in time. In addition, creatinine is affected by multiple extra-renal factors such as age, sex, race, body volume, muscle catabolism, protein intake, gastrointestinal hemorrhage and drugs. It can be seen that the increase in serum creatinine often lags behind the deterioration of renal functions and cannot accurately reflect the change of renal functions. Therefore, it is critical to find highly sensitive and specific biomarkers for early diagnosis and prognosis evaluation of AKI.

Long-chain non-coding RNA is a non-coding RNA with a length of more than 200 nucleotides. Research shows that the expression of the long-chain non-coding RNA becomes abnormal during the occurrence and development of various diseases so as to reflect the development and prognosis of diseases, and the expression of the long-chain non-coding RNA in blood is relatively stable. Therefore, the long-chain non-coding RNA has the advantages of high specificity, sensitivity, rapidness, convenience and pertinence as a molecular marker.

### SUMMARY

A first objective of the present invention is to provide a molecular marker for early detection of sepsis acute kidney injury, which is of great significance for early diagnosis and prediction of sepsis acute kidney injury.

A molecular marker TCONS_00024536 for early diagnosis and prediction of sepsis acute kidney injury, the sequence thereof being shown in SEQ ID NO: 1.

A second objective of the present invention is to provide application of the molecular marker TCONS_00024536, that is, the application of a product for detecting the expression level of TCONS_00024536 in preparing tools for early diagnosis and prediction of sepsis acute kidney injury.

The product comprises a preparation for detecting the expression level of TCONS_00024536 by RT-PCR or real-time quantitative PCR.

Primer sequences for specific amplification of TCONS_00024536 to detect the expression level of TCONS_00024536 by RT-PCR are as follows:

F:
5′-TAGGAAGGGCTGTTGACTGG-3′
R:
5′-CTGGGAGCTGGATTCAGAAG-3′.

A third objective of the present invention is to provide a kit for early diagnosis and prediction of sepsis acute kidney injury, which comprises a reagent for detecting the expression level of TCONS_00024536 by RT-PCR or real-time quantitative PCR.

The kit for early diagnosis and prediction of sepsis acute kidney injury comprises a pair of primers for specific amplification of TCONS_00024536 by RT-PCR, and primer sequences thereof are as follows:

F:
5′-TAGGAAGGGCTGTTGACTGG-3′
R:
5′-CTGGGAGCTGGATTCAGAAG-3′.

Beneficial Effects

The applicant found that the expression of TCONS_00024536 was up-regulated in patients with sepsis acute kidney injury (FIG. 2A-2B), suggesting that TCONS_00024536 is a molecular marker for diagnosis and prediction of sepsis acute kidney injury. The present invention provides a strong molecular biological basis for diagnosis and prediction of sepsis acute kidney injury, and has profound clinical significance and popularization.

### BRIEF DESCRIPTION OF THE DRAWINGS

FIG. 1A-1F show a result of chip expression profile analysis of LncRNA.

FIG. 1A. heat map; FIG. 1B. LncRNA up-regulated in sepsis AKI compared with control group and sepsis non-AKI compared with control group; FIG. 1C. LncRNA up-regulated and down-regulated in sepsis AKI compared with sepsis non-AKI; FIG. 1D. LncRNA down-regulated in sepsis AKI compared with control group and sepsis non-AKI compared with control group;

FIG. 1E. LncRNA up-regulated in sepsis non-AKI compared with control group while down-regulated in sepsis AKI compared with control group, and LncRNA up-regulated in sepsis non-AKI compared with control group while down-regulated in sepsis AKI compared with control group; FIG. 1F. TCONS_00024536 up-regulated in sepsis AKI compared with control group and down-regulated in sepsis non-AKI compared with control group.

FIG. 2A-2B show RT-PCR verifying the expression of TCONS_00024536 in control, sepsis AKI and sepsis non-AKI, wherein FIG. 2A shows TCONS_00024536 and internal reference GAPDH, and FIG. 2B is gray analysis. Difference is statistically significant.

### DESCRIPTION OF THE EMBODIMENTS

The following embodiments are intended to further illustrate the present invention rather than limiting the present invention.

### Embodiment 1: Screening Molecular Markers Associated with Sepsis Acute Kidney Injury

1. Sample Collection

Blood samples of healthy people, sepsis non-AKI patients and sepsis AKI patients were collected.

2. Preparation and Quality Analysis of RNA Sample

Total RNA was extracted using Trizol from Kangwei Century Company, specifically comprising the following steps:

1) 2 mL of whole blood was taken from a test tube treated with sodium citrate and put into an enzyme-free centrifuge tube;

2) plasma collection: centrifuging was performed at 3000 rpm for 10 min, and a supernatant (plasma) was carefully sucked from the top of the sample and put into another enzyme-free centrifuge tube;

3) 250 μL of plasma liquid was taken and transferred to a 1.5 ml centrifuge tube containing 750 μL of Trizol reagent, and the tube was shaken manually and violently till even mixing;

4) the homogenized sample was incubated at 15-30° C. for 5 min;

5) 0.2 mL of chloroform was added to the homogenized sample and the tube was covered tightly by a tube cap, incubation was performed at 15-30° C. for 2-3 min after manually shaking the tube violently for 15 sec, and centrifuging was performed at 4° C. and 12,000 rpm for 15 min;

6) the mixed liquid after centrifugation was divided into a red phenol chloroform phase in the lower layer and a colorless aqueous phase in the middle layer and the upper lay, RNA was completely distributed in the aqueous phase, and 500 μL of aqueous phase was sucked and transferred to a new centrifuge tube;

7) 500 μL of isopropyl alcohol was added to the new centrifuge tube and the mixture was evenly mixed to precipitate RNA therein, incubation was performed at 15-30° C. for 10 min after even mixing, and then centrifuging was performed at 4° C. and 12,000 rpm for 10 min;

8) the supernatant was removed, at least 1 mL of 75% ethanol was added, the RNA precipitate was washed, and centrifuging was performed at 4° C. and 7,500 rpm for 5 min after shaking;

9) the ethanol solution was removed and the RNA precipitate was dried in the air for 5-10 min;

(10) 20 μL of RNA-free enzyme water was added, blowing was performed several times with a pipette, and then the centrifuge tube containing RNA was covered and stored in a refrigerator of −80° C.; and

11) RNA quality analysis: the concentration and purity of the extracted RNA were measured using NanoDrop® ND-1000.

3. High-Throughput Transcriptome Sequencing

1) RNA-seq read localization

2) Transcription abundance assessment

3) Detection of differentially expressed genes

4. Results

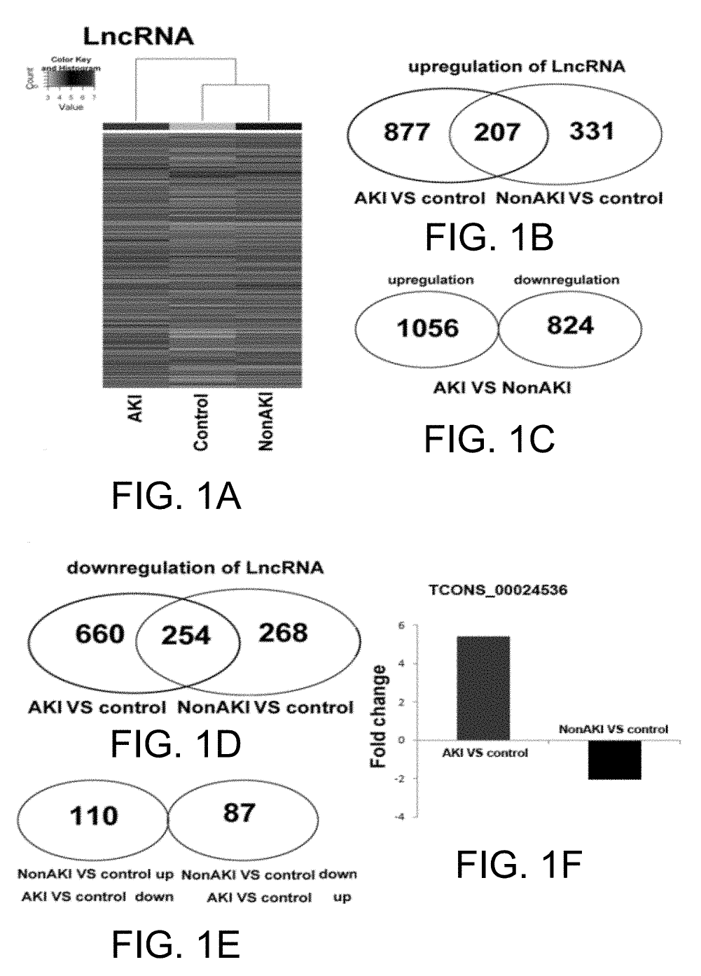

The RNA-seq results are shown in FIG. 1A-1F. Plasma from 5 healthy controls, 15 sepsis non-AKI patients and 15 sepsis AKI patients were studied using an LncRNA chip in a pilot experiment. It can be known from the analysis of microarray results that:

(1) compared with healthy people, the expression of 1084 lncRNAs was up-regulated and the expression of 914 lncRNAs was down-regulated in blood samples of sepsis AKI patients; (2) compared with healthy people, the expression of 538 lncRNAs was up-regulated and the expression of 522 lncRNAs was down-regulated in plasma of sepsis non-AKI patients; (3) compared with sepsis non-AKI, the expression of 1,056 lncRNAs was up-regulated and the expression of 824 lncRNAs was down-regulated in sepsis AKI; (4) compared with the control group, the expression of 207 lncRNAs was up-regulated and the expression of 254 lncRNAs was down-regulated in both sepsis non-AKI and sepsis AKI; (5) the expression of 110 lncRNAs was up-regulated in sepsis non-AKI compared with the control group and down-regulated in sepsis AKI compared with the control group, and the expression of 87 lncRNAs was down-regulated in sepsis non-AKI compared with the control group and up-regulated in sepsis AKI compared with the control group; and (6) TCONS_00024536 was up-regulated by 5.2 times in sepsis AKI compared with the control group and down-regulated by 2 times in sepsis non-AKI compared with the control group. The gene chip results show that LncRNA expression is changed in both sepsis AKI and non-AKI. LncRNAs up-regulated in AKI and down-regulated in non-AKI were finally screened through comparison of various groups, with the focus on TCONS_00024536 which significantly changes, suggesting that LncRNA can be used as a marker.

In order to further verify the results of the chip, the expression of TCONS_00024536 by RT-PCR was further verified.

### Embodiment 2: RT-PCR Verification of Differential Expression

1. According to the detection results of high-throughput sequencing, RT-PCR verification was selected. Blood samples of healthy people, sepsis AKI patients and sepsis non-AKI patients were collected according to the sample collection method in Embodiment 1.

2. The RNA extraction procedure was the same as that in Embodiment 1.

3. Reverse transcription: a reverse transcription kit from ThermoFish was used.

| RNA | 0.1 ng-5 μg | |
|---|---|---|
| Primer: Oligo (dT) 18 primer, or | 1 | μL |
| Random Hexamer primer, or | 1 | μL |
| gene-specific primer | 15-20 | pmol |

| | |
|---|---|
| Enzyme-free water | Add to 12 μL |

Place on the Eppendorf PCR instrument to incubate at 65° C. for 5 min,
then put on ice immediately

| | | |
|---|---|---|
| 5X Reaction Buffer | 4 | μL |
| RiboLock RNase Inhibitor (20 U/μL) | 1 | μL |
| 10 mM dNTP Mix | 2 | μL |
| Revert Aid M-MuLV RT (200 U/μL) | 1 | μL |
| Total volume | 20 | μL |

Ionize an EP tube,
Place on the Eppendorf PCR instrument to incubate at 25° C. for 5 min,
then incubate at 42° C. for 60 min, finally incubate at 70° C. for 5 min
to end the reaction

4. RT-PCR amplification

1) Primer Design

According to the gene coding sequences of TCONS_00024536 and Gapdh, PCR amplification primers were designed and synthesized by Sangon Biotech in Shanghai. The specific primer sequences are as follows:

TCONS_00024536:

F:
5′-TAGGAAGGGCTGTTGACTGG-3′
R:
5′-CTGGGAGCTGGATTCAGAAG-3′

Gapdh gene:

F:
5′-CAAGGTCATCCATGACAACTTTG-3′
R:
5′-GTCCACCACCCTGTTGCTGTAG-3′

2) A PCR reaction system was prepared according to the following table (wherein the Tap MasterMix premix system was purchased from Kangwei Century Company):

| Reagent | 20 μL reaction system | |
|---|---|---|
| 2X Tap MasterMix | 10 | μL |
| Forward Primer, 10 μM | 2 | μL |
| Reverse Primer, 10 μM | 2 | μL |
| Template DNA | 2 | μL |
| ddH₂O | 4 | μL |

PCR reaction procedure

| Step | Temperature | Time | |
|---|---|---|---|
| Initial denaturation | 94° C. | 2 | min |
| Denaturation | 94° C. | 30 | s |
| Annealing | 55° C. | 30 | s |
| Extension | 72° C. | 30 | s |
| Final extension | 72° C. | 2 | min |

Denaturing and annealing were repeated for 35-40 cycles.

5. Agarose gel electrophoresis: 10 μL of PCR product was taken for electrophoresis detection on 2% agarose gel, agarose electrophoresis photos of RT-PCR results were subjected to gray scanning by an image analysis system, and the gray scanning value (optical density scanning value) was IA, which represented the brightness of a target band on the gel, reflecting the amount of the target band.

Relative amount of PCR product (IA ratio)=IA of target fragment/IA of internal control GAPDH

The IA ratio of cDNA fragments in the healthy control group, the sepsis AKI group and the sepsis non-AKI group was calculated by the above formula, and the ratio was the relative amount of the RT-PCR product.

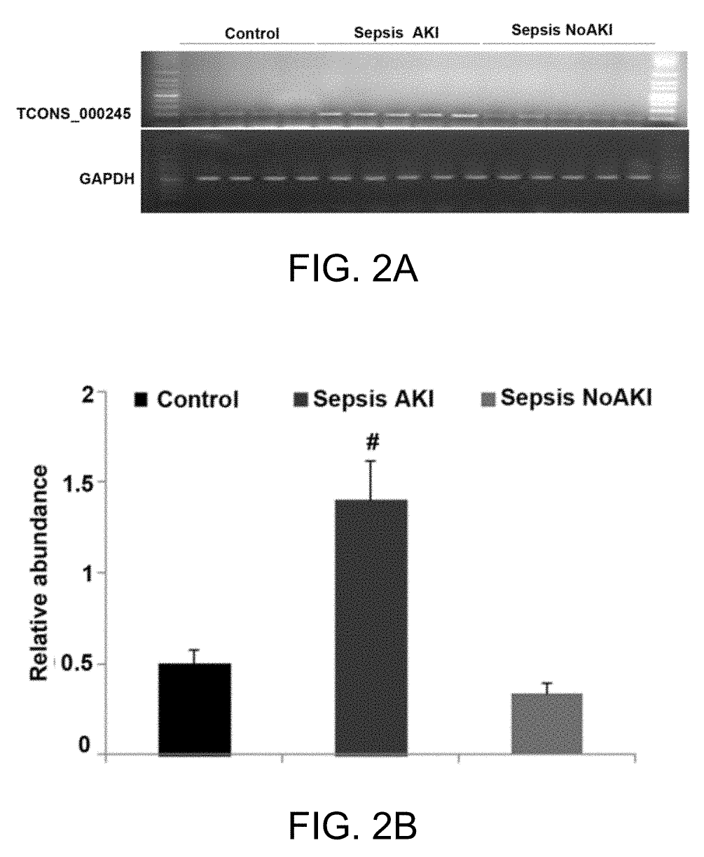

6. Results: as shown in FIG. 2A-2B, the expression of TCONS_00024536 in the blood of sepsis AKI patients was up-regulated compared with healthy people and the sepsis non-AKI group. RT-PCR results verified the discovery of the LncRNA chip, suggesting that TCONS_00024536 can be used as an early diagnostic molecular marker for sepsis AKI.

The above description of the embodiments is at least used for understanding the method of the present invention and the core idea thereof. Several improvements and modifications can be made to the present invention without departing from the principle of the present invention, and these improvements and modifications will also fall within the protection scope of the claims of the present invention.

What is claimed is:

1. A long non-coding RNA (LncRNA) molecule TCONS_00024536 having a nucleotide sequence as set forth in SEQ ID NO: 1, for use as a molecular marker for diagnosis and/or prediction of sepsis acute kidney injury.

2. A method for diagnosing and/or predicting a sepsis acute kidney injury in a patient, the method comprising:
providing a test sample of blood obtained from the patient;
preparing an RNA sample from the test sample;
detecting the expression level of the LncRNA molecule TCONS_00024536 of claim 1 in the RNA sample; and
using the detected level of expression of the LncRNA molecule TCONS_00024536 to diagnose and/or predict sepsis acute kidney injury in the patient.

3. The method according to claim 2, wherein the detection of the expression level of the LncRNA molecule TCONS_00024536 is performed by RT-PCR or real-time quantitative PCR.

4. The method according to claim 2, wherein prior to detecting the expression level of the LncRNA molecule TCONS_00024536, the LncRNA molecule is subjected to amplification by RT-polymerase chain reaction (RT-PCR) using a pair of primers specific to the LncRNA molecule, wherein the pair of primers comprises a first primer having a nucleotide sequence as set forth in SEQ ID NO: 2 and a second primer having a nucleotide sequence as set forth in SEQ ID NO: 3.

5. A method for diagnosing and/or predicting a sepsis acute kidney injury in a patient, the method comprising:
providing a test sample of blood obtained from the patient;
preparing an RNA sample from the test sample;
detecting the expression level of the LncRNA molecule TCONS_00024536 of claim 1 in the RNA sample; and
comparing the expression level with a control expression level of the LncRNA molecule TCONS_00024536 present in an RNA sample which is prepared from a blood sample obtained from a healthy patient,
wherein a difference in the expression level as compared to the control expression level is used for diagnosing and/or predicting sepsis acute kidney injury in the patient.

6. The method according to claim 5, wherein a higher level of expression of the LncRNA molecule TCONS_00024536 when compared to the control expression level, correlates with the presence of a sepsis acute kidney injury.

7. The method according to claim 5, wherein the detection of the expression level of the LncRNA molecule TCONS_00024536 is performed by RT-PCR or real-time quantitative PCR.

8. The method according to claim 5, wherein prior to detecting the expression level of the LncRNA molecule TCONS_00024536, the LncRNA molecule is subjected to amplification by RT-polymerase chain reaction (RT-PCR) using a pair of primers specific to the LncRNA molecule, wherein the pair of primers comprises a first primer having a nucleotide sequence as set forth in SEQ ID NO: 2 and a second primer having a nucleotide sequence as set forth in SEQ ID NO: 3.

9. A kit for diagnosis and/or prediction of sepsis acute kidney injury in a patient, comprising a reagent for detecting the expression level of the LncRNA molecule TCONS_00024536 of claim 1, wherein the detection is performed by RT-PCR or real-time quantitative PCR.

10. The kit according to claim 9, in which the reagent comprises a pair of primers for specific amplification of the LncRNA molecule TCONS_00024536, wherein the pair of primers comprises a first primer having a nucleotide sequence as set forth in SEQ ID NO: 2 and a second primer having a nucleotide sequence as set forth in to SEQ ID NO: 3.

11. A reagent for detecting the expression level of the LncRNA molecule TCONS_00024536 of claim 1, wherein the detection is made by RT-PCR or real-time quantitative PCR, the reagent comprising a pair of primers for specific amplification of the LncRNA molecule, wherein the pair of primers comprises a first primer having a nucleotide sequence as set forth in SEQ ID NO: 2 and a second primer having a nucleotide sequence as set forth in SEQ ID NO: 3.

12. The reagent according to claim 9, wherein the reagent is used in detecting the expression level of the LncRNA molecule TCONS_00024536 according to the method of claim 2, in which detecting the expression level of the LncRNA molecule is made by RT-PCR or real-time quantitative PCR.

13. The reagent according to claim 9, wherein the reagent is used in detecting the expression level of the LncRNA molecule TCONS_00024536 according to the method of claim 5, in which detecting the expression level of the LncRNA molecule is made by RT-PCR or real-time quantitative PCR.


SEQUENCE LISTING (5 sequences)

SEQ ID NO: 1 - RNA, 1611 nt, Homo sapiens
cuuuuuuuucccccugagauggagucucacucuaucgcccaggcuggagugcggugcaguggcauggucuuggcucacugcaaccuccaccuccuggguucaagcaauucuccugccucagccccccaaguagcugggacuauaggcaugccccaccacgcccagauuauuuuuguauuuuuaguagagacaggguuucaccauauuggccaggcuggucucgaacuccugaccucaagaucugcccaccuuggccucccaaagugcugggauuacaggcgugagcuacuaugcccagccuguccaccucuuccuuuagcaaacaaaaucccugggcgaccagagugaccccagaaauuggaggagggcagagaccccgucuuuggaggugauguggaggguguccaggcccgcccuugucucacaggcgcacacccugcuggaggccaagugucggacaugaaggugacgcugccccaugcuguccagacacccagcaaugcucagcacccggaaacaggaccggccucugcucugcguggagcccacccggacaguccaucucagccccuucuacccagggacccccaggccccugcccgcaugcaggaacuguccucugggcagagcagcccccuccccaugagggucucgcaggcccaggacucgcguccagcagcuccuggacauggccuugcugcccggccagggcuaguguccgugaacagagaauagugugugugagcggcucugucaucugucacuuggggcugccugugucccucuccaggaccucagugccgaucgcaggaaaggauguuggcacggcccccggugggacaggccagccacguuaggggaugcugcacgacaaagcccacggcgguggugucacaugaaugucaccugcucuucacacccagccuaggaagggcuguugacuggaggcugacauacccagcgaggccucugacgucccugucacccccuggggugggagaguccccucuuccagccugucuggggccuugccccucccuacagcccagcucgggaaacaaaugcuccuuccagcagcagcagagagcuguagagccggggcuccgggcagcuucugaauccagcucccaggccacagaucacagggacaggacgggagccucccacacagacgccaggcagcaggaccccggcugggaagucacauccaaaggacagaaccaccugcccaaguccccuccacuucugagaaccaccugcccaaguccccuccacgucugagaaccaccugcccaaguccccuccacaucugaacacucauugcuguguccggacgcagaaauucacacucggauuuuguccggcauucccacucccugcccugcuuggagaggcaggaccgcaggucugccggccccaacggcagcgccacagugcccggcacagaggccugcuggucgcgcauacaaagcacccgccggccauuaucauuccggagccggccagagccucccggcugcaucucaucggucauucagcagcccguaaugacuugucaguuccuugcccugccauuuucugucuaauuaauucuuuuauugugcaccggau

SEQ ID NO: 2 - DNA, 20 bp, Artificial Sequence
  Synthesized Primer F for specific amplification of TCONS_00024536
taggaagggctgttgactgg

SEQ ID NO: 3 - DNA, 20 bp, Artificial Sequence
  Synthesized Primer R for specific amplification of TCONS_00024536
ctgggagctggattcagaag

SEQ ID NO: 4 - DNA, 23 bp, Artificial Sequence
  Synthesized Primer F for specific amplification of Gapdh gene
caaggtcatccatgacaactttg

SEQ ID NO: 5 - DNA, 22 bp, Artificial Sequence
  Synthesized Primer R for specific amplification of Gapdh gene
gtccaccaccctgttgctgtag